(19) United States
(12) Patent Application Publication
(10) Pub. No.: US 2026/0154791 A1
(43) Pub. Date: Jun. 4, 2026

(54) PHOTOMETRIC IMAGE ENHANCEMENT FOR ENDOSCOPY

(71) Applicant: Auris Health, Inc., Santa Clara, CA (US)

(72) Inventors: Mali Shen, Sunnyvale, CA (US); Hedyeh Rafii-Tari, Mountain View, CA (US); Saif Iftekar Sayed, Foster City, CA (US); Morgan Jill Ringel, San Francisco, CA (US)

(73) Assignee: Auris Health, Inc., Santa Clara, CA (US)

(21) Appl. No.: 19/396,249

(22) Filed: Nov. 20, 2025

Related U.S. Application Data
(60) Provisional application No. 63/727,144, filed on Dec. 2, 2024.

(51) Int. Cl. G06T 5/70; A61B 34/00; A61B 34/20; G06T 5/60

(52) U.S. Cl. CPC G06T 5/70; A61B 34/20; A61B 34/25; G06T 5/60; G06T 2200/24; G06T 2207/10068; G06T 2207/20084; G06T 2207/30061; G06T 2207/30084

(57) ABSTRACT

This disclosure provides methods, devices, and systems for navigating medical instruments. The present implementations more specifically relate to photometric image enhancement techniques for endoscopy. In some aspects, a machine learning model may be trained to infer an enhanced image from a low-quality image captured by the camera of an endoscope. As used herein, the term “low-quality image” refers to any image containing visual artifacts, obstructions, and/or other deficiencies. By contrast, an “enhanced image” is a digitally modified representation of a low-quality image that removes and/or corrects at least some of the visual artifacts, obstructions, or other deficiencies in the low-quality image. A controller for a medical system may extract information from the enhanced image based on one or more image processing operations and generate a graphical user interface (GUI) for navigating the instrument within the anatomy based at least in part on the information extracted from the enhanced image data.

### CROSS-REFERENCE TO RELATED APPLICATIONS

This application claims priority and benefit under 35 U.S.C. § 119(e) to U.S. Provisional Patent Application No. 63/727,144 , filed Dec. 2, 2024, which is incorporated herein by reference in its entirety.

### TECHNICAL FIELD

This disclosure relates generally to medical systems, and specifically to photometric image enhancement for endoscopy.

### DESCRIPTION OF RELATED ART

Many medical procedures involve a series of complex steps that require careful movement and positioning of medical tools or instruments inside a patient's body (such as a flexible catheter or endoscope having a camera disposed on its distal tip). Some medical procedures can be performed, at least in part, by a robotic system or apparatus, which can aid a medical provider (such as a physician or a technician) in navigating or positioning medical instruments. For example, to remove urinary stones from the bladder and ureter, the medical provider can insert a ureteroscope into the urinary tract through the urethra. A ureteroscope includes an endoscope at its distal end configured to enable visualization of the urinary tract.

The medical provider can control a robotic system to advance and navigate the ureteroscope from the urethra, through the bladder, up the ureter, and into the kidney where the kidney stone is located. The robotic system may include, or may be coupled to, one or more display devices that can provide information to assist the physician in navigating the medical instrument. Such information can be captured or obtained using various sensors disposed on or otherwise coupled to the robotic system.

Images or video captured by an endoscope are often used for navigating and/or guiding medical instruments (such as a bronchoscope, ureteroscope, or percutaneous access needle, among other examples) to a target object or position within the anatomy (such as the location of a kidney stone or lung nodule). Instrument navigation and/or target localization systems generally rely on clear and unobstructed views from within the anatomy. However, the images captured by an endoscope often contain visual artifacts or other obstructions which may corrupt or otherwise render the images unsuitable for such intended uses. Thus, there is a need to improve the quality of images or video captured by an endoscope within an anatomy.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method for controlling a medical system. The method includes steps of receiving image data captured by a camera disposed on a distal end of an instrument inserted within an anatomy; inferring enhanced image data from the received image data based on a neural network model trained to filter visual artifacts or obstructions from image data; extracting information from the enhanced image data based on one or more image processing operations; and generating a graphical user interface (GUI) for navigating the instrument within the anatomy based at least in part on the information extracted from the enhanced image data.

Another innovative aspect of the subject matter of this disclosure can be implemented in a controller for a medical system, including a processing system and a memory. The memory stores instructions that, when executed by the processing system, cause the controller to receive image data captured by a camera disposed on a distal end of an instrument inserted within an anatomy; infer enhanced image data from the received image data based on a neural network model trained to filter visual artifacts or obstructions from image data; extract information from the enhanced image data based on one or more image processing operations; and generate a graphical user interface (GUI) for navigating the instrument within the anatomy based at least in part on the information extracted from the enhanced image data.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows a more detailed example of the robotic system of FIG. 1, according to some implementations.

FIG. 3 shows a more detailed example of the control system of FIG. 1, according to some implementations.

FIG. 4 shows a block diagram of an example mapping system, according to some implementations.

FIG. 5 shows a block diagram of an example image processing pipeline, according to some implementations.

FIG. 6 shows an example image enhancer for bronchoscopic image data, according to some implementations.

FIG. 7 shows an example image enhancer for urological image data, according to some implementations.

FIG. 8 shows a block diagram of an example machine learning system, according to some implementations.

FIG. 9 shows a block diagram of an example controller for a medical system, according to some implementations.

FIG. 10 shows an illustrative flowchart depicting an example operation for controlling a medical system, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations.

Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, some medical systems utilize images or video captured by an endoscope (also referred to as endoscopic “vision”) for navigating and/or guiding medical instruments (such as a bronchoscope, a ureteroscope, or a percutaneous access needle) to a target object or position within an anatomy (such as the location of a kidney stone or lung nodule). Vision-related tasks, such as instrument navigation or target localization, generally rely on clear and unobstructed views from within the anatomy. However, the images captured by the camera of an endoscope may contain visual artifacts or other obstructions which can corrupt or otherwise render the images unsuitable for such vision-related tasks. For example, the camera's field of view (FOV) can be occluded or otherwise obstructed by various objects within the anatomy, such as mucus, blood, stone dust, bubbles, and/or other medical instruments. Fluid flowing within the FOV can also cause motion blur in images captured by the camera. Further, changes in lighting conditions (such as due to changes in position and/or orientation of a light source associated with the endoscope) can result in specular reflection artifacts, camera saturation, and over-or under-exposure in various regions of an image.

Aspects of the present disclosure recognize that machine learning can be used to improve or enhance the quality of images captured by the camera of an endoscope, for example, by reducing the presence of artifacts and/or removing obstructions that would otherwise cause the images to be unusable for various vision-related tasks (such as instrument navigation and/or target localization). Machine learning is a technique for improving the ability of a computer system to perform a certain task. Machine learning generally comprises a training phase and an inferencing phase. During the training phase, a machine learning system is provided with one or more “answers” (also referred to as “ground truth”) and a large volume of raw training data associated with the answers. The machine learning system analyzes the training data to learn a set of rules (also referred to as a “machine learning model”) that can be used to describe each of the answers. During the inferencing phase, the machine learning system may infer answers from new data using the learned set of rules.

In some aspects, a machine learning model may be trained to infer an enhanced image from a low-quality image captured by the camera of an endoscope. As used herein, the term “low-quality image” refers to any image containing visual artifacts, obstructions, and/or other deficiencies that render the image unsuitable for certain vision-related tasks associated with a medical system (such as instrument navigation and/or target localization). By contrast, an “enhanced image” is a digitally modified representation of a low-quality image that removes and/or corrects at least some of the visual artifacts, obstructions, or other deficiencies present in the original low-quality image. In some implementations, the machine learning model may be trained to infer the enhanced image using generative image inpainting techniques. In some other implementations, the machine learning model may be trained to infer the enhanced image using super resolution techniques. Still further, in some implementations, the machine learning model may be trained to infer the enhanced image using generative style transfer techniques.

Aspects of the present disclosure may be used to perform robotic-assisted medical procedures, such as endoscopic access, percutaneous access, or treatment for a target anatomical site. For example, robotic tools may engage or control one or more medical instruments (such as an endoscope) to access a target site within a patient's anatomy or perform a treatment at the target site. In some implementations, the robotic tools may be guided or controlled, at least in part, by a human operator (such as a physician or a technician). In some other implementations, the robotic tools may operate in an autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as an endoscope that is exclusively controlled and operated by a physician). The systems and techniques described herein also may be applicable beyond the context of medical procedures (such as in simulated environments or laboratory settings, such as with models or simulators, among other examples).

Although certain aspects of the present disclosure are described in detail herein in the context of renal, urological, or nephrological procedures, such as kidney stone removal and treatment procedures, it should be understood that such context is provided for convenience and clarity, and the concepts disclosed herein are applicable to any suitable medical procedure.

However, as mentioned, description of the renal or urinary anatomy and associated medical issues and procedures is presented herein to aid in the description of the concepts disclosed herein. In some implementations, the techniques and systems described herein are discussed in the context of a percutaneous procedure, which can include any procedure where access is gained to a target location by making a puncture or incision in the skin, mucous membrane, or other body layer. However, it should be understood that these techniques and systems can be implemented in the context of any endoscopic procedure, including bronchoscopy, ureteroscopy, gastroscopy, nephroscopy, and nephrolithotomy, among other examples.

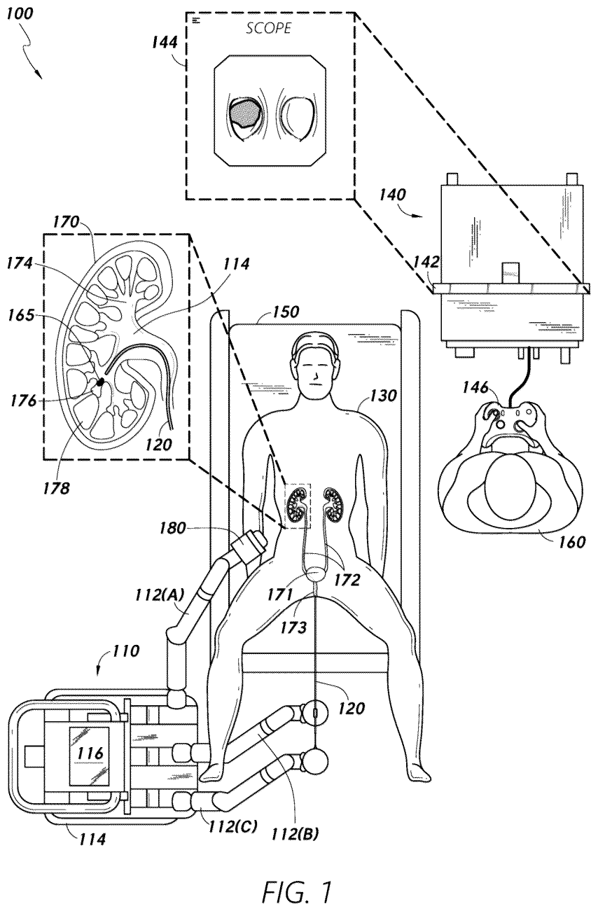

FIG. 1 shows an example medical system 100, according to some implementations. In some implementations, the medical system 100 may be used for surgical and/or diagnostic procedures. The medical system 100 includes a robotic system 110 configured to engage with and/or control a medical instrument 120 to perform a procedure on a patient 130. The medical system 100 also includes a control system 140 configured to interface with the robotic system 110, provide information regarding the procedure, and/or perform a variety of other operations. For example, the control system 140 can include a display 142 to present a user interface 144 to assist the physician 160 in using the medical instrument 120. Further, the medical system 100 can include a table 150 configured to hold the patient 130 and/or an imaging sensor 180, such as a camera, x-ray, computed tomography (CT), magnetic resonance imaging (MRI), positron emission tomography (PET) device, or the like.

In some implementations, the physician may perform a minimally-invasive medical procedure, such as a ureteroscopy. The physician 160 can interact with the control system 140 to control the robotic system 110 to navigate the medical instrument 120 (such as a basket retrieval device and/or scope) from the urethra up to the kidney 170 where the stone 165 is located. The control system 140 can provide information via a display 142 regarding the medical instrument 120 to assist the physician 160 in navigation, such as real-time images from the medical instrument 120 or the imaging sensor 180. Once at the site of the kidney stone, the medical instrument 120 can be used to break-up and/or capture a urinary stone 165.

In some implementations of using the medical system 100, a physician 160 can perform a percutaneous procedure. To illustrate, if the patient 130 has a kidney stone 165 in a kidney 170 that is too large to be removed through a urinary tract, the physician 160 can perform a procedure to remove the kidney stone through a percutaneous access point on the patient 130. For example, the physician 160 can interact with the control system 140 to control the robotic system 110 to navigate the medical instrument 120 (such as a scope) from the urethra up to the kidney 170 where the stone 165 is located. The control system 140 can provide information via a display 142 regarding the medical instrument 120 to assist the physician 160 in navigating the medical instrument 120, such as real-time images from the medical instrument 120 or the imaging sensor 180. Once at the site of the kidney stone, the medical instrument 120 can be used to designate a target location for a second medical instrument (not shown) to access the kidney percutaneously (such as a desired point to access the kidney). To minimize damage to the kidney, the physician 160 can designate a particular papilla as the target location for entering into the kidney with the second medical instrument. However, other target locations can be designated or determined. Once the second medical instrument has reached the target location, the physician 160 can use the second medical instrument and/or another medical instrument to extract the kidney stone from the patient 130, such as through the percutaneous access point.

In the example of FIG. 1, the medical instrument 120 is implemented as a scope (also referred to as an “endoscope” or “ureteroscope”). However, other example suitable medical instruments may include a basket retrieval device, a needle, a catheter, a guidewire, a lithotripter, forceps, a vacuum, and a scalpel, among other examples. In some implementations, a medical instrument is a steerable device, while other implementations a medical instrument is a non-steerable device. As used herein, a “surgical tool” refers to a device that is configured to puncture or to be inserted through the human anatomy, such as a needle, a scalpel, a guidewire, and so on. However, a surgical tool can refer to other types of medical instruments. In some implementations, multiple medical instruments may be used. For example, an endoscope can be used with a basket retrieval device. In some implementations, the medical instrument 120 may be a compound device incorporating several instruments, such as a vacuum, a basket retrieval device, a scope or various combinations of instruments.

The robotic system 110 can be configured to at least partly facilitate a medical procedure. The robotic system 110 can be arranged in a variety of ways depending on the particular procedure. The robotic system 110 can include one or more robotic arms 112 (robotic arms 112(a), 112(b), 112(c)) to engage with and/or control the medical instrument 120 to perform a procedure. As shown, each robotic arm 112 can include multiple arm segments coupled to joints, which can provide multiple degrees of movement. In the example of FIG. 1, the robotic system 110 is positioned proximate to the patient's 130 lower torso and the robotic arms 112 are actuated to engage with and position the medical instrument 120 for access into an access point, such as the urethra of the patient 130. With the robotic system 110 properly positioned, the medical instrument 120 can be inserted into the patient 130 robotically using the robotic arms 112, manually by the physician 160, or a combination thereof.

The robotic system 110 can also include a base 114 coupled to the one or more robotic arms 112. The base 114 can include a variety of subsystems, such as control electronics, a power source, pneumatics, an optical source, an actuator (such as motors to move the robotic arm), control circuitry, memory, and/or a communication interface. In some implementations, the base 114 includes an input/output (I/O) device 116 configured to receive input, such as user input to control the robotic system 110, and provide output, such as patient status, medical instrument location, or the like. The I/O device 116 can include a controller, a mouse, a keyboard, a microphone, a touchpad, other input devices, or combinations of the above. The I/O device can include an output component, such as a speaker, a display, a haptic feedback device, other output devices, or combinations of the above. In some implementations, the robotic system 110 is movable (such as the base 114 includes wheels) so that the robotic system 110 can be positioned in a location that is appropriate or desired for a procedure. In other implementations, the robotic system 110 is a stationary system. Further, in some implementations, the robotic system 110 is integrated into the table 150.

The robotic system 110 can be coupled to any component of the medical system 100, such as the control system 140, the table 150, the imaging sensor 180, and/or the medical instruments 120. In some implementations, the robotic system is communicatively coupled to the control system 140. In one example, the robotic system 110 can receive a control signal from the control system 140 to perform an operation, such as to position a robotic arm 112 in a particular manner, manipulate a scope, and so on. In response, the robotic system 110 can control a component of the robotic system 110 to perform the operation. In another example, the robotic system 110 can receive an image from the scope depicting internal anatomy of the patient 130 and/or send the image to the control system 140 (which can then be displayed on the control system 140). Further, in some implementations, the robotic system 110 is coupled to a component of the medical system 100, such as the control system 140, to receive data signals, power, and so on. Other devices, such as other medical instruments, intravenous bags, blood packs or the like can also be coupled to the robotic system 110 or other components of the medical system 100 depending on the medical procedure being performed.

The control system 140 can be configured to provide various functionality to assist in performing a medical procedure. In some implementations, the control system 140 can be coupled to the robotic system 110 and operate in cooperation with the robotic system 110 to perform a medical procedure on the patient 130. For example, the control system 140 can communicate with the robotic system 110 via a wireless or wired connection (such as to control the robotic system 110, the medical instrument 120, and/or to receive an image(s) captured by a scope), control the flow of fluids through the robotic system 110 via one or more fluid channels, provide power to the robotic system 110 via one or more electrical connections, provide optical signals to the robotic system 110 via one or more optical fibers or other components. Further, in some implementations, the control system 140 can communicate with a scope to receive sensor data. Moreover, in some implementations, the control system 140 can communicate with the table 150 to position the table 150 in a particular orientation or otherwise control the table 150.

As shown in FIG. 1, the control system 140 includes various I/O devices configured to assist the physician 160 or others in performing a medical procedure. In some implementations, the control system 140 includes an input device 146 that is employed by the physician 160 or another user to control the medical instrument 120. For example, the input device 146 can be used to navigate the medical instrument 120 within the patient 130. The physician 160 can provide input via the input device 146 and, in response, the control system 140 can send control signals to the robotic system 110 to manipulate the medical instrument 120.

Although the input device 146 is illustrated as a controller in the example of FIG. 1, the input device 146 can be implemented as a variety of types of I/O devices, such as a (touchscreen or touchpad, a mouse, a keyboard, a microphone, a smart speaker, etc. As also shown in FIG. 1, the control system 140 can include the display 142 to provide various information regarding a procedure. For example, the control system 140 can receive real-time images that are captured by a scope and display the real-time images via the display 142. Additionally, or alternatively, the control system 140 can receive signals (such as analog, digital, electrical, acoustic/sonic, pneumatic, tactile, or hydraulic signals) a medical monitor and/or a sensor associated with the patient 130, and the display 142 can present information regarding the health of the patient 130 and/or an environment of the patient 130. Such information can include information that is displayed via a medical monitor including, for example, a heart rate (such as an electrocardiogram (ECG) or heart rate variability (HRV)), blood pressure and/or rate, muscle bio-signals (such as electromyography (EMG)), body temperature, oxygen saturation (such as SpO2), carbon dioxide (CO2), brainwave (such as electroencephalogram (EEG)), and environmental temperature, among other examples.

FIG. 1 also shows various anatomy of the patient 130 relevant to certain aspects of the present disclosure. In particular, the patient 130 includes kidneys 170 fluidly connected to a bladder 171 via ureters 172, and a urethra 173 fluidly connected to the bladder 171. As shown in the enlarged depiction of the kidney 170, the kidney includes calyxes 174 (such as major and minor calyxes), renal papillae (including the renal papilla 176, also referred to as “the papilla 176”), and renal pyramids (including the renal pyramid 178). In these examples, a kidney stone 165 is located in proximity to the papilla 176. However, the kidney stone can be located at other locations within the kidney 170.

As shown in FIG. 1, to remove the kidney stone 165 in the example minimally-invasive procedure, the physician 160 can position the robotic system 110 at the foot of the table 150 to initiate delivery of the medical instrument 120 into the patient 130. In particular, the robotic system 110 can be positioned within proximity to a lower abdominal region of the patient 130 and aligned for direct linear access to the urethra 173 of the patient 130. From the foot of the table 150, the robotic arm 112(B) can be controlled to provide access to the urethra 173. In this example, the physician 160 inserts the medical instrument 120 at least partially into the urethra along this direct linear access path (also referred to as “a virtual rail”). The medical instrument 120 can include a lumen configured to receive the scope and/or basket retrieval device, thereby assisting in insertion of those devices into the anatomy of the patient 130.

Once the robotic system 110 is properly positioned and/or the medical instrument 120 is inserted at least partially into the urethra 173, the scope can be inserted into the patient 130 robotically, manually, or a combination thereof. For example, the physician 160 can connect the medical instrument 120 to the robotic arm 112(C). The physician 160 can then interact with the control system 140, such as the input device 146, to navigate the medical instrument 120 within the patient 130. For example, the physician 160 can provide input via the input device 146 to control the robotic arm 112(C) to navigate the medical instrument 120 through the urethra 173, the bladder 171, the ureter 172, and up to the kidney 170.

The control system 140 can include various components (also referred to as “subsystems”) to facilitate its functionality. Example suitable subsystems include control electronics, a power source, pneumatics, an optical source, an actuator, control circuitry, memory, and/or a communication interface. In some implementations, the control system 140 includes a computer-based control system that stores executable instructions, that when executed, implement various operations. In some implementations, the control system 140 is movable, such as that shown in FIG. 1, while in other implementations, the control system 140 is a stationary system. Although various functionality and components are discussed as being implemented by the control system 140, any of this functionality and/or components can be integrated into and/or performed by other systems and/or devices, such as the robotic system 110 and/or the table 150.

The medical system 100 can provide a variety of benefits, such as providing guidance to assist a physician in performing a procedure (such as instrument tracking or patient status), enabling a physician to perform a procedure from an ergonomic position without the need for awkward arm motions and/or positions, enabling a single physician to perform a procedure with one or more medical instruments, avoiding radiation exposure (such as associated with fluoroscopy techniques), enabling a procedure to be performed in a single-operative setting, and providing continuous suction to remove an object more efficiently (such as to remove a kidney stone). Further, the medical system 100 can provide non-radiation-based navigational and/or localization techniques to reduce physician exposure to radiation and/or reduce the amount of equipment in an operating room. Moreover, the medical system 100 can divide functionality into the control system 140 and the robotic system 110, each of which can be independently movable. Such division of functionality and/or movability can enable the control system 140 and/or the robotic system 110 to be placed at locations that are optimal for a particular medical procedure, which can maximize working area around the patient, and/or provide an optimized location for a physician to perform a procedure. For example, many aspects of the procedure can be performed by the robotic system 110 (which is positioned relatively close to the patient) while the physician manages the procedure from the comfort of the control system 140 (which can be positioned farther way).

In some implementations, the control system 140 can function even if located in a different geographic location from the robotic system 110. For example, in a tele-health implementation, the control system 140 is configured to communicate over a wide area network with the robotic system 110. In one scenario, a physician 160 may be located in one hospital with the control system 140 while the robotic system 110 is located in a different hospital. The physician may then perform the medical procedure remotely. This can be beneficial where remote hospitals, such as those in rural areas, have limited expertise in particular procedures.

Those hospitals can then rely on more experienced physicians in other locations. In some implementations, a control system 140 is able to pair with a variety of robotic systems 110, for example, by selecting a specific robotic system and forming a secure network connection (such as using passwords, encryption, or authentication tokens). Thus, a physician in one location may be able to perform medical procedures in a variety of different locations by setting up a connection with robotic systems 110 located at each of those different locations.

In some implementations, the robotic system 110, the table 150, the medical instrument 120, the needle and/or the imaging sensor 180 are communicatively coupled to each other over a network, which can include a wireless and/or wired network. Example networks include one or more personal area networks (PANs), one or more local area networks (LANs), one or more wide area networks (WANs), one or more Internet area networks (IANs), one or more cellular networks, the Internet, etc. Further, in some implementations, the control system 140, the robotic system 110, the table 150, the medical instrument 120, and/or the imaging sensor 180 are connected for communication, fluid exchange, gas exchange, and/or power exchange, via one or more support cables.

Although not illustrated in FIG. 1, in some implementations, the medical system 100 includes and/or is associated with a medical monitor configured to monitor health of the patient 130 and/or an environment in which the patient 130 is located. For example, a medical monitor can be located in the same environment where the medical system 100 is located, such as within an operating room. The medical monitor can be physically and/or electrically coupled to one or more sensors that are configured to detect or determine one or more physical, physiological, chemical, and/or biological signals, parameters, properties, states and/or conditions associated with the patient 130 and/or the environment. For example, the one or more sensors can be configured to determine or detect any type of physical properties, including temperature, pressure, vibration, haptic or tactile features, sound, optical levels or characteristics, load or weight, flow rate (such as of target gases and/or liquid), amplitude, phase, and/or orientation of magnetic and electronic fields, constituent concentrations relating to substances in gaseous, liquid, or solid form.

The one or more sensors can provide the sensor data to the medical monitor and the medical monitor can present information regarding the health of the patient 130 and/or the environment of the patient 130. Such information can include information that is displayed via a medical monitor including, for example, a heart rate (such as ECG or HRV), blood pressure and/or rate, muscle bio-signals (such as EMG), body temperature, oxygen saturation (such as SpO2), CO2, brainwave (such as EEG), and environmental temperature, among other examples. In some implementations, the medical monitor and/or the one or more sensors are coupled to the control system 140 and the control system 140 is configured to provide information regarding the health of the patient 130 and/or the environment of the patient 130.

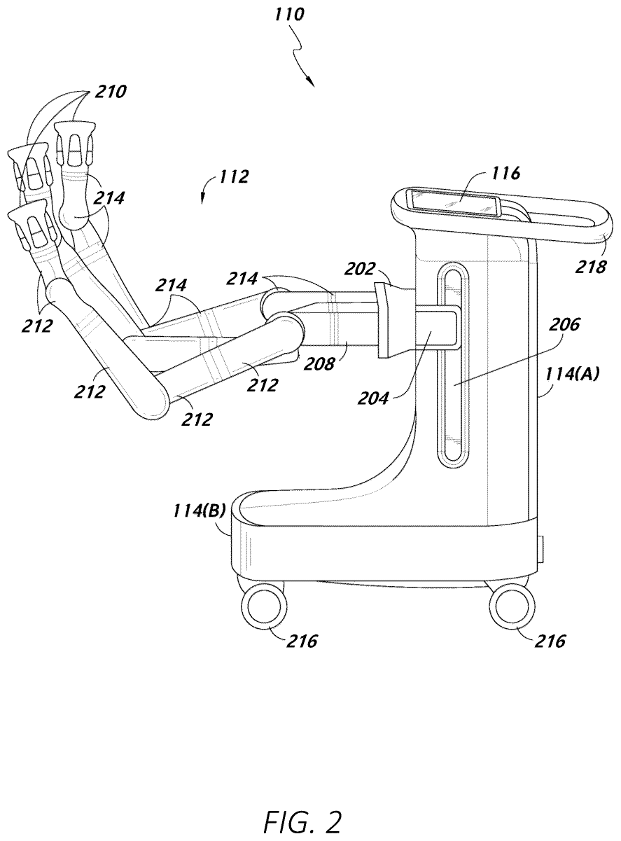

FIG. 2 shows a more detailed example of the robotic system 110 of FIG. 1, according to some implementations. In the example of FIG. 2, the robotic system 110 is illustrated as a cart based robotically-enabled system that is movable. However, the robotic system 110 can be implemented as a stationary system and/or integrated into a table in some other implementations.

The robotic system 110 can include the support structure 114 including an elongated section 114(A) (also referred to as a “column”) and a base 114(B). The column 114(A) can include one or more carriages, such as a carriage 202 (also referred to as an “arm support”) for supplying the deployment of one or more the robotic arms 112 (such as the 3 arms shown in FIG. 2). The carriage 202 can include individually configurable arm mounts that rotate along a perpendicular axis to adjust the base of the robotic arms 112 for positioning relative to a patient. The carriage 202 also includes a carriage interface 204 that allows the carriage 202 to vertically translate along the column 114(A). The carriage interface 204 is connected to the column 114(A) through slots, such as slot 206, that are positioned on opposite sides of the column 114(A) to guide the vertical translation of the carriage 202. The slot 206 includes a vertical translation interface to position and hold the carriage 202 at various vertical heights relative to the base 114(B). Vertical translation of the carriage 202 allows the robotic system 110 to adjust the reach of the robotic arms 112 to meet a variety of table heights, patient sizes, and/or physician preferences. Similarly, the individually configurable arm mounts on the carriage 202 allow a robotic arm base 208 of the robotic arms 112 to be angled in a variety of configurations. The column 114(A) can internally comprise mechanisms, such as gears and/or motors, that are designed to use a vertically aligned lead screw to translate the carriage 202 in a mechanized fashion in response to control signals generated in response to user inputs, such as inputs from the I/O device(s) 116.

In some implementations, the slot 206 can be supplemented with a slot cover(s) that is flush and/or parallel to the slot surface to prevent dirt and/or fluid ingress into the internal chambers of the column 114(A) and/or the vertical translation interface as the carriage 202 vertically translates. The slot covers can be deployed through pairs of spring spools positioned near the vertical top and bottom of the slot 206. The covers can be coiled within the spools until deployed to extend and retract from their coiled state as the carriage 202 vertically translates up and down. The spring-loading of the spools can provide force to retract the cover into a spool when the carriage 202 translates towards the spool, while also maintaining a tight seal when the carriage 202 translates away from the spool. The covers can be connected to the carriage 202 using, for example, brackets in the carriage interface 204 to ensure proper extension and retraction of the covers as the carriage 202 translates.

The base 114(B) can balance the weight of the column 114(A), the carriage 202, and/or arms 112 over a surface, such as the floor. Accordingly, the base 114(B) can house heavier components, such as one or more electronics, motors, and/or power supply, as well as components that enable movement and/or immobilize the robotic system 110. For example, the base 114(B) can include rollable wheels 216 (also referred to as “casters”) that allow for the robotic system 110 to move around the room for a procedure. After reaching an appropriate position, the casters 216 can be immobilized using wheel locks to hold the robotic system 110 in place during the procedure. As shown, the robotic system 110 also includes a handle 218 to assist with maneuvering and/or stabilizing the robotic system 110.

The robotic arms 112 can generally comprise robotic arm bases 208 and end effectors 210, separated by a series of linkages 212 that are connected by a series of joints 214. Each joint 214 can comprise an independent actuator and each actuator can comprise an independently controllable motor. Each independently controllable joint 214 represents an independent degree of freedom available to the robotic arm 112. For example, each of the arms 112 can have seven joints, and thus, provide seven degrees of freedom. However, any number of joints can be implemented with any degrees of freedom. In examples, a multitude of joints can result in a multitude of degrees of freedom, allowing for “redundant” degrees of freedom.

Redundant degrees of freedom allow the robotic arms 112 to position their respective end effectors 210 at a specific position, orientation, and/or trajectory in space using different linkage positions and/or joint angles. In some implementations, the end effectors 210 can be configured to engage with and/or control a medical instrument, a device, or object. Such freedom of movement can allow the robotic system 110 to position and/or direct a medical instrument from a desired point in space and/or allow a physician to move the arms 112 into a clinically advantageous position away from the patient to create access, while avoiding arm collisions.

As shown in FIG. 2, the robotic system 110 can also include the I/O device(s) 116. The I/O device(s) 116 can include a display, a touchscreen, a touchpad, a projector, a mouse, a keyboard, a microphone, a speaker, a controller, a camera (such as to receive gesture input), or another I/O device to receive input and/or provide output. The I/O device(s) 116 can be configured to receive touch, speech, gesture, or any other type of input. The I/O device(s) 116 can be positioned at the vertical end of column 114(A) (such as the top of the column 114(A)) and/or provide a user interface for receiving user input and/or for providing output. For example, the I/O device(s) 116 can include a touchscreen (such as a dual-purpose device) to receive input and provide a physician with pre-operative and/or intra-operative data. Example pre-operative data can include pre-operative plans, navigation, and/or mapping data derived from pre-operative CT scans, and/or notes from pre-operative patient interviews. Example intra-operative data can include optical information provided from a tool or instrument, sensor, and/or coordinate information from sensors, as well as vital patient statistics, such as respiration, heart rate, and/or pulse. The I/O device(s) 116 can be positioned and/or tilted to allow a physician to access the I/O device(s) 116 from a variety of positions, such as the side of the column 114(A) opposite the carriage 202. From this position, the physician can view the I/O device(s) 116, the robotic arms 112, and/or a patient while operating the I/O device(s) 116 from behind the robotic system 110.

The robotic system 110 can include a variety of other components. For example, the robotic system 110 can include one or more control electronics/circuitry, power sources, pneumatics, optical sources, actuators (such as motors to move the robotic arms 112), memory, and/or communication interfaces (such as to communicate with another device). In some implementations, the memory can store computer-executable instructions that, when executed by the control circuitry, cause the control circuitry to perform any of the operations discussed herein. For example, the memory can store computer-executable instructions that, when executed by the control circuitry, cause the control circuitry to receive input and/or a control signal regarding manipulation of the robotic arms 112 and, in response, control the robotic arms 112 to be positioned in a particular arrangement and/or to navigate a medical instrument connected to the end effectors 210.

In some implementations, robotic system 110 is configured to engage with and/or control a medical instrument, such as the medical instrument 120. For example, the robotic arms 112 can be configured to control a position, orientation, and/or tip articulation of a scope (such as a sheath and/or a leader of the scope). In some implementations, the robotic arms 112 can be configured/configurable to manipulate the scope using elongate movement members. The elongate movement members can include one or more pull wires (such as pull or push wires), cables, fibers, and/or flexible shafts. To illustrate, the robotic arms 112 can be configured to actuate multiple pull wires coupled to the scope to deflect the tip of the scope. Pull wires can include any suitable or desirable materials, such as metallic and/or non-metallic materials. Example suitable materials include stainless steel, Kevlar, tungsten, or carbon fiber, among other examples. In some implementations, the scope is configured to exhibit nonlinear behavior in response to forces applied by the elongate movement members. The nonlinear behavior can be based on stiffness and compressibility of the scope, as well as variability in slack or stiffness between different elongate movement members.

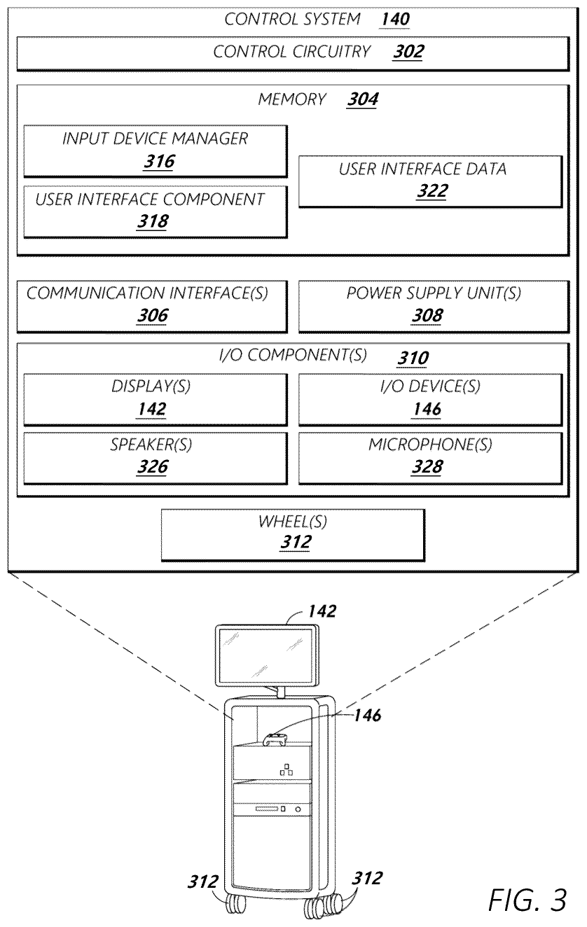

FIG. 3 shows a more detailed example of the control system 140 of FIG. 1, according to some implementations. As shown in FIG. 3, the control system 140 can include one or more devices, modules, and/or units (also referred to as “components”), either separately or individually and/or in combination or collectively: control circuitry 302, data storage or memory 304, one or more communication interfaces 306, one or more power supply units 308, one or more I/O components 310, and/or one or more wheels 312 (such as casters or other types of wheels). In some implementations, the control system 140 can comprise a housing or enclosure configured and/or dimensioned to house or contain at least part of one or more of the components of the control system 140. In the example of FIG. 3, the control system 140 is illustrated as a cart-based system that is movable with the one or more wheels 312. After reaching the appropriate position, the one or more wheels 312 can be immobilized using wheel locks to hold the control system 140 in place. However, the control system 140 can be implemented as a stationary system, integrated into another system and/or device.

Although certain components of the control system 140 are illustrated in FIG. 3, it should be understood that additional components not shown can be included in implementations in accordance with the present disclosure. Furthermore, certain of the illustrated components can be omitted in some implementations. Although the control circuitry 302 is illustrated as a separate component in the diagram of FIG. 3, it should be understood that any or all of the remaining components of the control system 140 can be embodied at least in part in the control circuitry 302. That is, the control circuitry 302 can include various devices (active and/or passive), semiconductor materials and/or areas, layers, regions, and/or portions thereof, conductors, leads, vias, connections, and/or the like, wherein one or more of the other components of the control system 140 and/or portion(s) thereof can be formed and/or embodied at least in part by such circuitry components and/or devices.

The various components of the control system 140 can be electrically and/or communicatively coupled using certain connectivity circuitry, devices, and/or features, which may or may not be part of the control circuitry 302. For example, the connectivity feature(s) can include one or more printed circuit boards configured to facilitate mounting and/or interconnectivity of at least some of the various components or circuitry of the control system 140. In some implementations, two or more of the control circuitry 302, the data storage or memory 304, the communication interface(s) 306, the power supply unit(s) 308, and/or the I/O component(s) 310, can be electrically and/or communicatively coupled to each other.

As illustrated, the memory 304 can include an input device manager 316 and a user interface component 318 configured to facilitate various functionality discussed herein. In some implementations, the input device manager 316, and/or the user interface component 318 can include one or more instructions that are executable by the control circuitry 302 to perform one or more operations. Although many implementations are discussed in the context of the components 316-318 including one or more instructions that are executable by the control circuitry 302, any of the components 316-318 can be implemented at least in part as one or more hardware logic components, such as one or more application specific integrated circuits (ASIC), one or more field-programmable gate arrays (FPGAs), one or more program-specific standard products (ASSPs), one or more complex programmable logic devices (CPLDs), and/or the like. Furthermore, although the components 316-318 are illustrated as being included within the control system 140, any of the components 316-318 can be implemented at least in part within another device/system, such as the robotic system 110, the table 150, or another device/system. Similarly, any of the other components of the control system 140 can be implemented at least in part within another device/system.

The input device manager 316 can be configured to receive inputs from the input device 146 and translate them into actions performable by the robotic system 110. The user interface component 318 can be configured to facilitate one or more user interfaces (also referred to as a “graphical user interface” or “GUI”). For example, the user interface component 318 can provide user interface data 322 for display to the user. The communication interfaces 306 can be configured to communicate with one or more devices, sensors, and/or systems. For example, the one or more communication interfaces 306 can send and/or receive data in a wireless and/or wired manner over a network. A network in accordance with implementations of the present disclosure can include a LAN, WAN (such as the Internet), PAN, or body area network (BAN), among other examples. In some implementations, the one or more communication interfaces 306 can implement a wireless technology such as Bluetooth, Wi-Fi, or near field communication (NFC), among other examples.

The one or more power supply units 308 can be configured to manage power for the control system 140 and/or the robotic system 110. In some implementations, the one or more power supply units 308 include one or more batteries, such as a lithium-based battery, a lead-acid battery, an alkaline battery, and/or another type of battery. That is, the one or more power supply units 308 can comprise one or more devices and/or circuitry configured to provide a source of power and/or provide power management functionality. Moreover, in some implementations the one or more power supply units 308 include a mains power connector that is configured to couple to an alternating current (AC) or direct current (DC) mains power source.

The one or more I/O components 310 can include a variety of components to receive input and/or provide output, such as to interface with a user. The one or more I/O components 310 can be configured to receive touch, speech, gesture, or any other type of input. In examples, the one or more I/O components 310 can be used to provide input regarding control of a device or system, such as to control the robotic system 110, navigate the scope or other medical instrument attached to the robotic system 110, or control the table 150. As shown, the one or more I/O components 310 can include the one or more displays 142 (also referred to as “display devices”) configured to display data. The one or more displays 142 can include one or more liquid-crystal displays (LCD), light-emitting diode (LED) displays, organic LED displays, plasma displays, electronic paper displays, and/or any other type(s) of technology. In some implementations, the one or more displays 142 include one or more touchscreens configured to receive input and/or display data. Further, the one or more I/O components 310 can include the one or more input devices 146, which can include a touchscreen, touch pad, controller, mouse, keyboard, wearable device (such as an optical head mounted display), or virtual or augmented reality device (such as head mounted display). Additionally, the one or more I/O components 310 can include one or more speakers 326 configured to output sounds based on audio signals and/or one or more microphones 328 configured to capture or record audio. In some implementations, the one or more I/O components 310 include or are implemented as a console.

Although not shown in FIG. 3, the control system 140 can include and/or control other components, such as one or more pumps, flow meters, valve controls, and/or fluid access components in order to provide controlled irrigation and/or aspiration capabilities to a medical instrument (such as a scope) and/or a device that can be deployed through a medical instrument. In some implementations, irrigation and aspiration capabilities can be delivered directly to a medical instrument through separate cable(s). Further, the control system 140 can include a voltage and/or surge protector designed to provide filtered and/or protected electrical power to another device, such as the robotic system 110, thereby avoiding placement of a power transformer and other auxiliary power components in robotic system 110, resulting in a smaller, more moveable robotic system 110.

The control system 140 can also include support equipment for sensors deployed throughout the medical system 100. For example, the control system 140 can include opto-electronics equipment for detecting, receiving, and/or processing data received from optical sensors and/or cameras. Such opto-electronics equipment can be used to generate real-time images for display in any number of devices/systems, including in the control system 140.

In some implementations, the control system 140 can be coupled to the robotic system 110, the table 150, and/or a medical instrument, such as the medical instrument 120, through one or more cables or connections (not shown). In some implementations, support functionality from the control system 140 can be provided through a single cable, simplifying and de-cluttering an operating room. In other implementations, specific functionality can be coupled in separate cabling and connections. For example, while power can be provided through a single power cable, the support for controls, optics, fluidics, and/or navigation can be provided through a separate cable.

The term “control circuitry” is used herein according to its broad and ordinary meaning, and can refer to any collection of one or more processors, processing circuitry, processing modules or units, chips, dies (such as semiconductor dies including come or more active and/or passive devices and/or connectivity circuitry), microprocessors, micro-controllers, digital signal processors, microcomputers, central processing units, graphics processing units, field programmable gate arrays, programmable logic devices, state machines (such as hardware state machines), logic circuitry, analog circuitry, digital circuitry, and/or any device that manipulates (analog and/or digital) signals based on hard coding of the circuitry and/or operational instructions. Control circuitry can further comprise one or more, storage devices, which can be embodied in a single memory device, a plurality of memory devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. It should be noted that in implementations in which control circuitry comprises a hardware state machine (and/or implements a software state machine), analog circuitry, digital circuitry, and/or logic circuitry, data storage device(s) or register(s) storing any associated operational instructions can be embedded within, or external to, the circuitry comprising the state machine, analog circuitry, digital circuitry, and/or logic circuitry.

The term “memory” is used herein according to its broad and ordinary meaning and can refer to any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s) or protocols, including any suitable or desirable computer-readable instructions, data structures, program modules, or other types of data.

Computer-readable media that can be implemented in accordance with implementations of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

In some aspects, the user interface component 318 may be configured to generate a three-dimensional (3D) model of an anatomy (also referred to as an “anatomical map”) that can help guide a user with navigating and/or positioning medical instruments relative to the anatomy. For example, the anatomical map may depict a spatial relationship between the medical instrument(s) and various features of the anatomy (such as the locations of kidney stones, calyxes, papillae, and/or the walls of the kidney). In some implementations, the user interface component 318 may generate or reconstruct the anatomical map based on 3D images of the anatomy. Example suitable imaging technologies include computed tomography (CT), X-ray, fluoroscopy, positron emission tomography (PET), PET-CT, CT angiography, cone beam CT (CBCT), three-dimensional rotational angiography (3DRA), single-photon emission CT (SPECT), magnetic resonance imaging (MRI), optical coherence tomography (OCT), and ultrasound, among other examples. For example, a CT scanner may acquire tomographic images (also referred to as “tomograms”) of a patient's kidneys during the preoperative phase for a PCNL procedure. A tomogram is a cross-section or slice of a 3D volume such that multiple tomograms can be stacked or combined to recreate the 3D volume.

In some other implementations, the user interface component 318 may generate the anatomical map based on two-dimensional (2D) images of the anatomy. Example suitable 2D images include pyelograms captured (with or without a contrast agent) by a fluoroscopic imaging device (such as a CBCT scanner). Still further, in some implementations, the user interface component 318 may generate the anatomical map based on sensor data tracking the position and/or movement of the scope 120. For example, a user may trace the anatomy using the scope 120 while the control system 140 tracks a pose (such as a position and/or orientation) of the scope 120 based on real-time sensor data received from one or more sensors disposed on the scope 120. The user interface component 318 may generate the sensor map by mapping the position and/or movement of the scope 120 (also referred to as a “sensor map”). Example suitable sensor technologies that can be used for generating a sensor map include, among other examples, electromagnetic (EM) sensors for tracking a pose of the instrument and cameras for visualization within the anatomy.

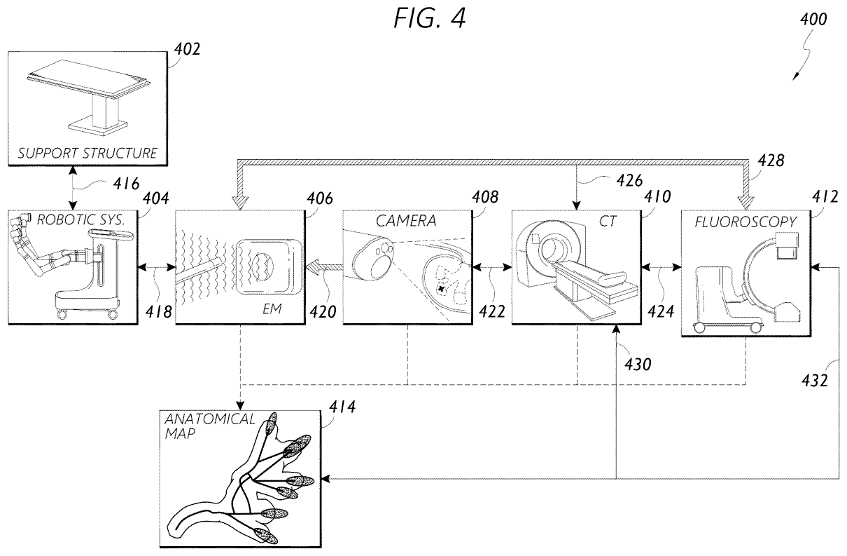

FIG. 4 shows a block diagram of an example mapping system 400, according to some implementations. The mapping system 400 includes various positioning or imaging systems or modalities 402-412 (also referred to as “subsystems”), which can be implemented to facilitate anatomical mapping, navigation, positioning, or visualization for procedures in accordance with one or more examples. For example, the various systems 402-412 can be configured to provide data for generating an anatomical map, determining a location of an instrument, determining a location of a target, and/or performing other techniques.

Each of the systems 402-412 can be associated with a respective coordinate space (also referred to as a “position coordinate frame”) or can provide data or information relating to instrument or anatomy locations, wherein registering the various coordinate spaces to one another can allow for integration of the various systems to provide mapping, navigation, or instrument visualization. As used herein, the term “registration” refers to a mapping or transformation between different coordinate spaces. For example, registering a first modality to a second modality can allow for determined positions in the first modality to be tracked or superimposed on or in a reference frame associated with the second modality, thereby providing layers of positional information that can be combined to provide a robust localization system.

In some aspects, the system 400 may be configured to perform one or more localization operations. As used herein, the term “localization” refers to a process or technique for determining a position (or location) and orientation (or heading), collectively referred to as the “pose” of an instrument or feature, within an anatomical space. In some implementations, the anatomical space in which a medical instrument can be localized (such as where a pose or shape of the instrument is determined or estimated) may be a 2D or 3D portion of a patient's tracheobronchial airways, vasculature, urinary tract, gastrointestinal tract, or any organ or space accessed via lumens. Various modalities can be implemented to provide images, representations, or models of the anatomical space. In some implementations, an imaging modality may be used to capture or acquire images of a patient's anatomy during a preoperative phase of a medical procedure. In some other implementations, an imaging modality may be used to capture or acquire images of a patient's anatomy during an intraoperative phase of the medical procedure.

The systems 402-412 can provide information for generating a 2D or 3D anatomical map 414. While a kidney map is shown as an example of the anatomical map 414, it will be understood that the anatomical map 414 can be of any interior region of a body (such as the lungs). In some implementations, the anatomical map 414 may include spatial or contextual information (also referred to as “localization information”) to help guide a user with navigating and/or positioning an instrument to reach a target within the anatomy. For example, the localization information may include an estimated position, orientation, and/or shape of the instrument. The localization information also may include a shape, boundary, eccentricity, texture, and/or position of the target. In some implementations, the anatomical map 414 and/or localization information may be displayed to a user during a medical procedure to assist the user in performing the procedure. For example, a visualization of a tracked instrument can be superimposed on the anatomical map 414 based on position or sensor data associated with the tracked medical instrument.

In the example of FIG. 4, the system 400 is shown to include a support structure 402 (such as a surgical bed or other patient positioning or support platform). For example, the support structure 402 includes a planar surface that contacts and supports the patient. In some implementations, the position of the support structure 402 may be known based on data maintained relating to the position of the support structure 402 within the surgical or procedure environment. In some other implementations, the position of the support structure 402 may be sensed or otherwise determined using one or more markers or an appropriate imaging or positioning modality.

The system 400 further includes a robotic system 404 (such as a robotic cart or other device or system including one or more robotic end effectors). In some implementations, the robotic system 404 may be one example of the robotic system 110 of FIGS. 1 and 2. Data relating to the position or state of robotic arms, actuators, or other components of the robotic system 404 can be known or derived from robotic command data or other robotic data relative to a coordinate frame of the robotic system 404. In some examples, reference frame registration 416 occurs between the support structure 402 and the robotic system 404, which can be a relatively coarse registration, in some implementations, based on robotic system or cart-set-up procedure (which can have any suitable or desirable scheme).

The system 400 further includes an EM sensor system 406, which can include an EM field generator and one or more EM sensors. An EM sensor can be disposed on a portion of an instrument that is tracked or controlled, such as a distal end or tip of the instrument or along a length of the instrument, or other elongate member (such as a working channel) disposed in a lumen of the instrument. In some implementations, the EM field generator may be mechanically coupled to the support structure 402 or the robotic system 404 so that registration or association 418 between the systems can be known or determined. In some other implementations, the registration 418 between the EM sensor system 406 and the robotic system 404 may be determined based on forward kinematics or field generator mount transform information. For example, the field generator can be mounted to the support structure 402 such that the position of the field generator is known relative to the robotic system positioning frame based on a known relationship between the position of the support structure 402 and the robotic system 404. The EM sensor system 406 can provide instrument pose or path information based on sensor readings associated with the instrument.

The system 400 further includes an optical camera system 408 including one or more cameras or other imaging devices configured to generate images of an anatomy within a visual field thereof (such as real-time image data) during a surgical procedure. In some implementations, registration 420 between the optical camera system 408 and the EM sensor system 406 may be achieved through identification of features having EM sensor data associated therewith (such as a medical instrument tip) in images generated by the optical camera system 408. For example, the registration 420 may include a hand-eye calibration matrix that maps nay point or vector in the camera space to a respective point or vector in the EM sensor space. In some other implementations, the registration 420 may be determined based at least in part on hand-eye interaction of the physician when viewing real-time camera images while the EM-sensor-equipped endoscope is navigating in the patient anatomy.

The system 400 further includes a CT imaging system 410 configured to generate CT image data representing tomograms of the anatomy, which can be performed preoperatively or intraoperatively. The CT imaging system 410 is generally used for scanning a relatively large volume. For example, the CT imaging system 410 can be used to generate preoperative imaging data for producing the anatomical map 414 or for path navigation planning. Image processing can be implemented for registration 422 of the CT image data with the camera image data generated by the optical camera system 408. For example, common features identified in both camera image data and CT image data can be used to relate the CT image frame to the camera image frame in space. The CT imaging system 410 also may be registered 426 to the EM sensor system 406 using various techniques. For example, a mechanical structure of the CT imaging system 410 may have a known physical transform or relationship with respect to a mounting position of the EM field generator of the EM sensor system 406. Such known relationship can be used to register the CT image space to the EM sensor space.

The system 400 can further include a fluoroscopy imaging system 412 configured to generate tomographic images (such as real-time X-ray images) of the surgical site. The fluoroscopy imaging system 412 is generally used for scanning a smaller volume compared to the CT imaging system 410. In some implementations, the fluoroscopy imaging system 412 may be one example of the imaging system 122 of FIG. 1. For example, the fluoroscopy imaging system 412 may include a CBCT scanner coupled to a C-arm. In some implementations, the fluoroscopy imaging system 412 may be used with a contrast agent introduced into the anatomy to generate image data representing patient anatomy or instrumentation. The fluoroscopy imaging system 412 can be registered 424 to the CT imaging system 410 using any suitable image processing techniques.

The fluoroscopy imaging system 412 can also be registered 428 to the EM sensor system 406 using various techniques. In some implementations, a mechanical structure of the fluoroscopy imaging system 412 (such as the C-arm instrumentation) may have a known physical transform or relationship with respect to a mounting position of the EM field generator of the EM sensor system 406. Such known relationship can be used to register the fluoroscopy image space to the EM sensor space. In some other implementations, the EM-to-fluoroscopy registration 428 may combine other modalities, in addition to EM sensing and fluoroscopy imaging. For example, the system 400 may use the registration 420 between the camera system 408 and the EM sensor system 406, as well as the positions of known anatomical features in images captured by the camera system 408 and images captured by the fluoroscopy imaging system 412, to register the fluoroscopy image space to the EM sensor space.

In the example of FIG. 4, the CT imaging system 410 and fluoroscopy imaging system 412 are illustrated as separated systems. However, in some other implementations, a single imaging system may perform the functions of both the CT imaging system 410 and fluoroscopy imaging system 412.

The position, shape, and/or orientation of an instrument, such as an endoscope, can be determined using any one or more of the systems 402-412, which can facilitate generation of graphical interface data representing the estimated pose and/or shape of the instrument relative to the anatomical map 414. The position, shape, and/or orientation of the instrument and/or the anatomical map 414 can be displayed on a display device, such as via the control system 140 or robotic system 110 of FIG. 1, or other device. In some implementations, the anatomical map 414 also may indicate a position of a target within the anatomy (such as a kidney stone or lung nodule) that has been designated for treatment.

Although the systems 402-412 have been described in a particular order, the operations or functions associated therewith can be performed in different orders. In some implementations, the systems 402-412 can be used in different ways. In some other implementations, registration can occur between different systems and/or modalities.

As described with reference to FIGS. 1-4, images captured by the camera system 408 can be used for navigating and/or guiding medical instruments (such as a bronchoscope, a ureteroscope, or a percutaneous access needle) to a target object or position within an anatomy (such as the location of a kidney stone or lung nodule). For example, the images can be displayed on a user interface (such as the user interface 144 of FIG. 1) to provide a real-time endoscopic view of the anatomy. The camera data can also be used to generate an anatomical map (such as the anatomical map 414 of FIG. 4) or determine spatial and/or contextual information associated therewith. For example, the camera data can be used to register various coordinate spaces associated with different sensor or imaging modalities (such as described with reference to FIG. 4) or to determine a position, distance, orientation, size, shape, and/or texture of various anatomical features.

Many vision-related tasks (such as instrument navigation and/or localization of anatomical features) generally rely on clear and unobstructed views from within the anatomy. However, the images captured by the camera system 408 often contain visual artifacts or other obstructions which can corrupt or otherwise render the images unsuitable for such vision-related tasks. For example, the camera's FOV can be occluded or otherwise obstructed by various objects within the anatomy, such as mucus, blood, stone dust, bubbles, and/or other medical instruments. Fluid flowing within the FOV can also cause motion blur in images captured by the camera. Further, changes in lighting conditions (such as due to changes in position and/or orientation of a light source associated with the endoscope) can result in specular reflection artifacts, camera saturation, and over-or under-exposure in various regions of an image.

In some implementations, the camera system 408 may include a stereoscopic camera and/or multiple cameras having different FOVs. In such implementations, the images captured by a secondary camera may be used for navigation and/or localization if the primary camera view becomes corrupted (such as due to artifacts and/or obstructions). However, disposing multiple cameras within an anatomy may be expensive and difficult to implement. Thus, in some other implementations, the images captured by the camera system 408 may be processed through an image processing pipeline that improves or enhances the quality of the images, for example, by reducing the presence of artifacts and/or removing obstructions that would otherwise cause the images to be unusable for various vision-related tasks.

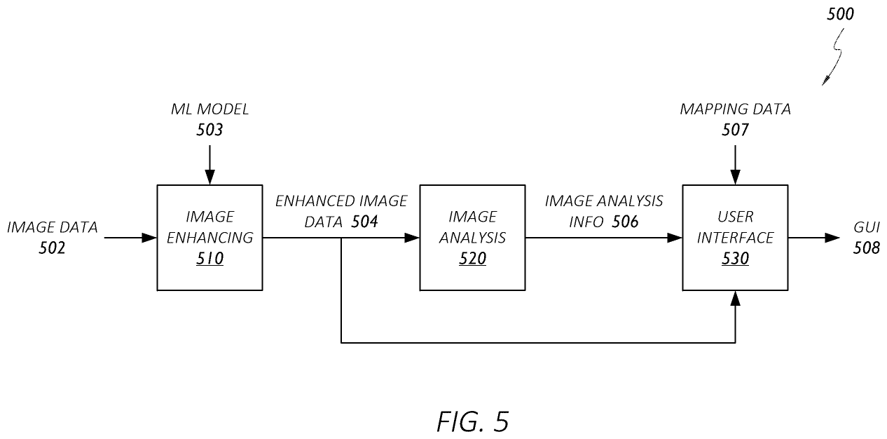

FIG. 5 shows a block diagram of an example image processing pipeline 500, according to some implementations. In some examples, the image processing pipeline 500 may be implemented by a controller associated with a medical system (such as the control system 140 of FIGS. 1 and 3). More specifically, the image processing pipeline 500 is configured to receive image data 502 captured by a camera associated with a medical instrument disposed within an anatomy (such as the scope 120 of FIG. 1) and generate a graphical user interface (GUI) 508, based at least in part on the image data 502, for navigating or guiding the medical instrument and/or another medical instrument (such as a percutaneous access needle) to reach or otherwise access a target within the anatomy (such as a kidney stone or lung nodule).

The image processing pipeline 500 includes an image enhancing component 510, an image analysis component 520, and a user interface component 530. The image enhancing component 510 is configured to digitally enhance the image data 502 using one or more image processing techniques. Example suitable image processing techniques include dehazing, deblurring, color correction, noise reduction, and glare reduction, among other examples. More specifically, the image enhancing component 510 is configured to produce enhanced image data 504 representing a higher quality image than the original image data 502.

As described with reference to FIG. 4, an endoscope may often capture low quality images from within the anatomy that contain visual artifacts, obstructions, or other deficiencies that can render the images unsuitable for various vision-related tasks (such as instrument navigation and/or localization of anatomical features). Thus, the image enhancing component 510 may improve the quality of the image data 502 by removing and/or correcting at least some of the visual artifacts, obstructions, or other deficiencies present in the original image data 502 so that the resulting enhanced image data 504 is better suited for at least some of the vision-related tasks.

In some aspects, the image enhancing component 510 may infer the enhanced image data 504 from the image data 502 using a machine learning (ML) model 503. In some implementations, the ML model 503 may be trained to infer the enhanced image data 504 using AI-based super resolution techniques. Super resolution is a technique for upscaling the resolution or pixel density of an image so that it appears sharper and/or contains more detail. Thus, super resolution can improve the quality of images with motion blur or high turbidity from mucus, blood, or other light-scattering fluids. For example, the ML model 503 can be trained to add details, textures, and edges into the original image data 502 while preserving fine elements and minimizing artifacts (such as jagged edges) in the resulting enhanced image data 504.

In some other implementations, the ML model 503 may be trained to infer the enhanced image data 504 using generative image inpainting techniques. Image inpainting is a technique for reconstructing or replacing corrupted regions of an image with corrected regions that are inferred based on the surrounding image information and context. For example, the ML model 503 can be trained to detect sections of the image data 502 that are noisy or corrupted and use generative image inpainting to fill in the corrupted regions of the image with corrected image data. In some implementations, the ML model 503 may leverage additional images of the anatomy (such as one or more CT scans acquired preoperatively) to ensure that the enhanced image data 504 preserves the structural integrity of the original image data 502.

In some other implementations, the ML model 503 may be trained to infer the enhanced image data 504 using generative style transfer techniques. Generative style transfer is a technique for generating the substance of one image in a specific context while preserving the essential features of the original image. For example, the ML model 503 can be trained to reconstruct the image data 502 in the style of a rendered CT scan. More specifically, the ML model 503 may combine the image data 502 with a CT scan of the anatomy (which may be acquired preoperatively) to produce an altered camera image in the style of a CT scan that has less noise than the original image data 502 but still captures valuable anatomical information.

Still further, in some implementations, the ML model 503 may be trained to infer the enhanced image data 504 using Neural Radiance Fields (NeRF) or Gaussian splatting techniques. Gaussian splatting and NeRF are techniques for creating 3D representations of a full scene from multiple frames of the scene. For example, the ML model 503 can be trained to reconstruct a 3D view of the anatomy based on multiple frames of image data 502. The image enhancing component 510 can then remove obstructions from the 3D view of the anatomy and render clean, unobstructed images based on the altered 3D view. In some implementations, the image enhancing component 510 may further improve the image quality of the rendered images by performing generative image inpainting on the altered 3D view using neighboring pixel data and/or preoperative image data (such as from a CT scan).

The image analysis component 520 is configured to extract image analysis information 504 from the enhanced image data 504. The image analysis information 506 may include any information that can be used for instrument navigation and/or localization of features within the anatomy. For example, the image analysis information 506 may include spatial or contextual information about various features that can be detected in the enhanced image data 504. Example suitable information includes, among other examples, an estimated position or orientation of a medical instrument, or a shape, boundary, eccentricity, texture, and/or position of an anatomical feature (such as a kidney stone or a lung nodule). In some implementations, the image analysis component 520 may extract the image analysis information 506 using various image processing techniques. Example suitable image processing techniques include segmentation, machine learning, and statistical analysis, among other examples.

The user interface component 530 is configured to generate the GUI 508 based at least in part on the enhanced image data 504 and/or the image analysis information 506. In some implementations, the GUI 508 may include a live endoscopic view of the anatomy (such as the user interface 144 of FIG. 1). In such implementations, the user interface component 530 may directly render or present the enhanced image data 504 in the live endoscopic view. In some other implementations, the GUI 508 may include an anatomical map (such as the anatomical map 414 of FIG. 4) depicting a spatial relationship between a medical instrument (such as the endoscope and/or a percutaneous access instrument) and the anatomy. In such implementations, the user interface component 530 may combine the image analysis information 506 with additional mapping data 507 (such as x-rays, pyelograms, tomograms, and/or EM sensor data) to map the pose of the instrument and/or the positions of various anatomical features to a 2D or 3D model of the anatomy generated based at least in part on the mapping data 507.

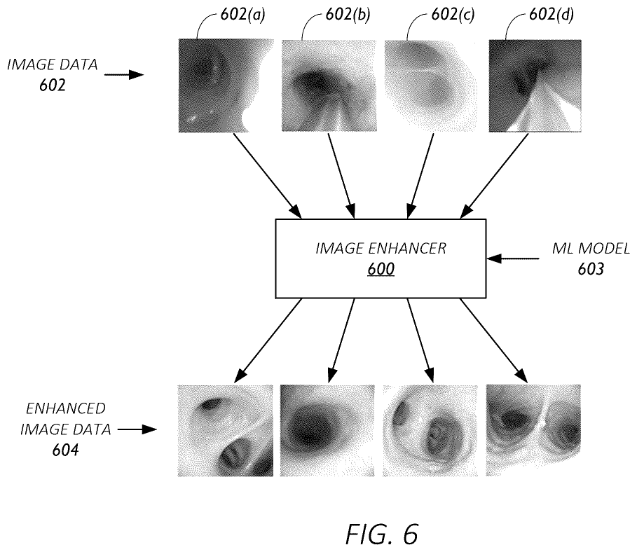

FIG. 6 shows an example image enhancer 600 for bronchoscopic image data, according to some implementations. In some implementations, the image enhancer 600 may be one example of the image enhancing component 510 of FIG. 5. More specifically, the image enhancer 600 is configured to generate enhanced image data 604 based on image data 602 captured by a camera associated with a medical instrument disposed within an anatomy (such as the endoscope 120 of FIG. 1). With reference to FIG. 5, the image data 602 may be one example of the image data 502 and the enhanced image data 604 may be one example of the enhanced image data 504.

The image data 602 may include visual artifacts, obstructions, and/or other deficiencies that render the image data 602 unusable for various vision-related tasks (such as instrument navigation and/or localization of anatomical features). In some implementations, the image enhancer 600 may digitally enhance low-quality image data 602 so that the resulting enhanced image data 604 is better suited for such vision-related tasks. FIG. 6 shows several example low-quality images 602(a)-602(d), containing visual artifacts and/or obstructions, captured by an endoscope disposed within a lung. More specifically, the low-quality images 602(a)-602(d) depict examples of specular reflection, instrument occlusion, motion blur, and obstructions due to mucus, respectively.

The image enhancer 600 is configured to enhance the quality of the image data 602 by performing one or more image processing operations. Example suitable image processing techniques include dehazing, deblurring, color correction, noise reduction, and glare reduction, among other examples. For example, the image enhancer 600 may use glare reduction and deblurring techniques to remove or reduce visual artifacts from the image data 602, such as specular reflections and/or blur (as shown in images 602(a) and 602(c)).

In some aspects, the image enhancer 600 may infer the enhanced image data 604 using an ML model 603. Example suitable ML models 603 include AI-based super resolution models, generative image inpainting models, generative style transfer models, and NeRF or Gaussian splatting models, among other examples. For example, the image enhancer 600 may use the ML model 603 to remove obstructions from the image data 602, such as due to the presence of instruments and/or mucus (as shown in images 602(b) and 602(d)). With reference to FIG. 5, the ML model 603 may be one example of the ML model 503.

FIG. 6 also shows several examples of enhanced image data 604 that can be produced by the image enhancer 600. In contrast to the low-quality images 602(a)-602(d), the enhanced image data 604 depicts clear and unobstructed views of the lung. Accordingly, the enhanced image data 604 may be usable for at least some vision-related tasks that the original image data 602 would otherwise be unusable for (such as instrument navigation and/or localization of anatomical features).

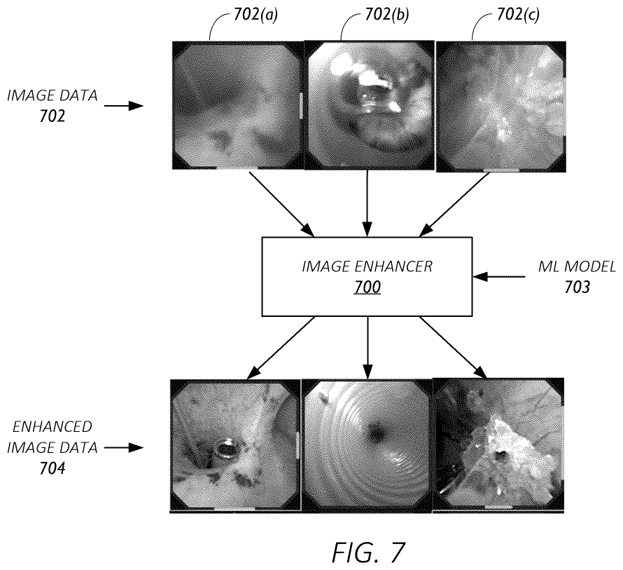

FIG. 7 shows an example image enhancer 700 for urological image data, according to some implementations. In some implementations, the image enhancer 700 may be one example of the image enhancing component 510 of FIG. 5. More specifically, the image enhancer 700 is configured to generate enhanced image data 704 based on image data 702 captured by a camera associated with a medical instrument disposed within an anatomy (such as the endoscope 120 of FIG. 1). With reference to FIG. 5, the image data 702 may be one example of the image data 502 and the enhanced image data 704 may be one example of the enhanced image data 504.

The image data 702 may include visual artifacts, obstructions, and/or other deficiencies that render the image data 702 unusable for various vision-related tasks (such as instrument navigation and/or localization of anatomical features). In some implementations, the image enhancer 700 may digitally enhance low-quality image data 702 so that the resulting enhanced image data 704 is better suited for such vision-related tasks. FIG. 7 shows several example low-quality images 702(a)-702(c), containing visual artifacts and/or obstructions, captured by an endoscope disposed within a kidney. More specifically, the low-quality images 702(a)-702(c) depict examples of motion blur (or changes in color), obstructions due to bubbles, and lasing artifacts (such as stone dust or fragments), respectively.

The image enhancer 700 is configured to enhance the quality of the image data 702 by performing one or more image processing operations. Example suitable image processing techniques include dehazing, deblurring, color correction, noise reduction, and glare reduction, among other examples. For example, the image enhancer 700 may use dehazing, deblurring, or color correction techniques to filter or remove visual artifacts associated with color changes and/or blur (as shown in image 702(a)). The image enhancer 700 also may use glare reduction techniques to filter or remove visual artifacts associated with bubbles (as shown in image 702(b)). Further, the image enhancer 700 may use noise reduction techniques to filter or remove visual artifacts associated with stone dust or fragments (as shown in image 702(c)).

In some aspects, the image enhancer 700 may infer the enhanced image data 704 using an ML model 703. Example suitable ML models 703 include AI-based super resolution models, generative image inpainting models, generative style transfer models, and NeRF or Gaussian splatting models, among other examples. For example, the image enhancer 700 may use the ML model 703 to remove obstructions from the image data 702, such as larger stone fragments and/or bubbles (as shown in images 702(b) and 702(c)). With reference to FIG. 5, the ML model 703 may be one example of the ML model 503.

FIG. 7 also shows several examples of enhanced image data 704 that can be produced by the image enhancer 700. In contrast to the low-quality images 702(a)-702(c), the enhanced image data 704 depicts clear and unobstructed views of the kidney. Accordingly, the enhanced image data 704 may be usable for at least some vision-related tasks that the original image data 702 would otherwise be unusable for (such as instrument navigation and/or localization of anatomical features).

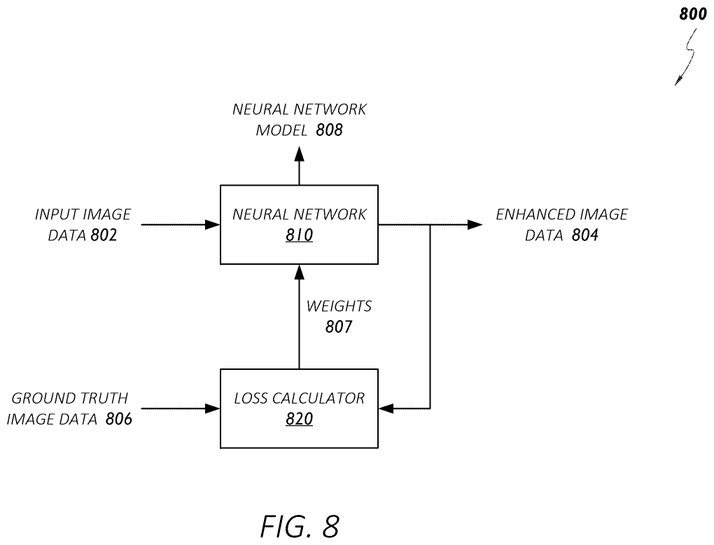

FIG. 8 shows a block diagram of an example machine learning system 800, according to some implementations. The machine learning system 800 is configured to produce a neural network model 808 based, at least in part, on input data 802 representing a large volume of images captured by cameras disposed on medical instruments (such as endoscopes) within an anatomy. With reference for example to FIGS. 1 and 3, the control system 140 may capture or acquire the input image data 802 via the endoscope 120 over one or more medical procedures. In some implementations, the neural network model 808 may be one example of any of the ML models 503, 603, or 703 of FIGS. 5-7, respectively. More specifically, the neural network model 808 may be trained to infer enhanced image data based on low-quality image data (such as described with reference to FIGS. 5-7).

The machine learning system 800 includes a neural network 810 and a loss calculator 820. In some aspects, the machine learning system 800 may train the neural network 810 to reproduce ground truth image data 806 based on the input image data 802. Deep learning is a particular form of machine learning in which the inferencing and training phases are performed over multiple layers. Deep learning architectures are often referred to as “artificial neural networks” due to the manner in which information is processed (similar to a biological nervous system). For example, each layer of an artificial neural network may be composed of one or more “neurons.” Each layer of neurons may perform a different transformation on the output data from a preceding layer so that the final output of the neural network results in the desired inferences. The set of transformations associated with the various layers of the network is referred to as a “neural network model.” Example suitable neural networks include convolutional neural networks (CNNs), recurrent neural networks (RNN), and long short-term memory (LSTM) networks, among other examples.

The ground truth image data 806 represents a desired output of the neural network 810 for a given set of input image data 802. Thus, in some implementations, the ground truth image data 806 may depict an enhanced image having fewer or no visual artifacts or obstructions compared to the original image data 802 provided as input to the neural network 810. The neural network 810 receives the input image data 802 and attempts to recreate the ground truth image data 806. For example, the neural network 810 may form a network of connections across multiple layers of artificial neurons that begin with the input image data 802 and lead to enhanced image data 804 at its output. The connections are weighted to result in enhanced image data 804 that closely resembles the ground truth image data 806.

The training operation is performed over multiple iterations. In each iteration, the neural network 810 produces enhanced image data 804 based on weighted connections across the layers of artificial neurons, and the loss calculator 820 updates the weights 807 associated with the connections based on an amount of loss (or error) between the enhanced image data 804 and the ground truth image data 806. The neural network 810 may output the weighted connections as the neural network model 808 when certain convergence criteria are met (such as when the loss falls below a threshold level or a predetermined number of training iterations have been performed).

In some aspects, the neural network 810 may implement an autoencoder architecture. An autoencoder is a type of artificial neural network that can be trained to reproduce, at its output, the same image received at its input. A bottleneck is imposed between the input layer and the output layer of the neural network, which reduces dimensionality of the outputs at the intermediate layers. As a result of the bottleneck, the autoencoder is forced to learn a compressed representation of the input image (also referred to as the “latent attributes” of the image). Thus, autoencoder architectures generally include an encoder component trained to convert a digital image into a lower-dimensional tensor or vector of latent attributes, and a decoder component trained to reconstruct the original image from the tensor or vector of latent attributes.

Unlike traditional autoencoders (which are trained to reproduce the same images at their outputs as received at their inputs), variational autoencoders (VAEs) can generate new output images that maintain the latent attributes of the original input images but are visually different than the input images. More specifically, VAEs are probabilistic models that use variational inference to generate the new output images by encoding a continuous, probabilistic representation of the latent space (rather than discrete, fixed representations of latent attributes). The decoder component samples from the latent space, between points representing the original latent attributes, to produce new images that resemble the original input images. Thus, the loss function associated with a VAE is quantified by a reconstruction loss (which represents the difference between the original input image data and the reconstructed image data) and its Kullback-Leibler (KL) divergence (which represents the divergence from the latent distribution).

Aspects of the present disclosure recognize that VAEs can capture the noise distribution differences between high-quality (or enhanced) images and low-quality images. For example, low-quality images may have a higher reconstruction error than high-quality images, and the lower layers of the neural network 810 may learn the noise distribution through training based on the loss function. Aspects of the present disclosure further recognize that conditional VAEs (CVAEs) are well-suited for generating enhanced images with improved image quality. CVAEs can produce new output images that are conditioned by specific inputs, for example, by adjusting the activations in lower layers. Thus, in some implementations, the neural network 810 may include a CVAE conditioned to produce enhanced image data 804 containing one or more desired anatomical features (such as to achieve any of the generative image inpainting, AI-based super resolution, or generative style transfer techniques described with reference to FIGS. 5-7).

Aspects of the present disclosure further recognize that the noise distribution learned by the neural network model 808 may be used for various other purposes in addition to, or in lieu of, generating enhanced image data. For example, in some implementations, the learned noise distribution may be used to classify images as low-quality or high-quality. In some other implementations, the neural network model 808 may be used to generate synthetic endoscopy images that can augment other datasets used in various simulation environments and/or for training other neural network models.

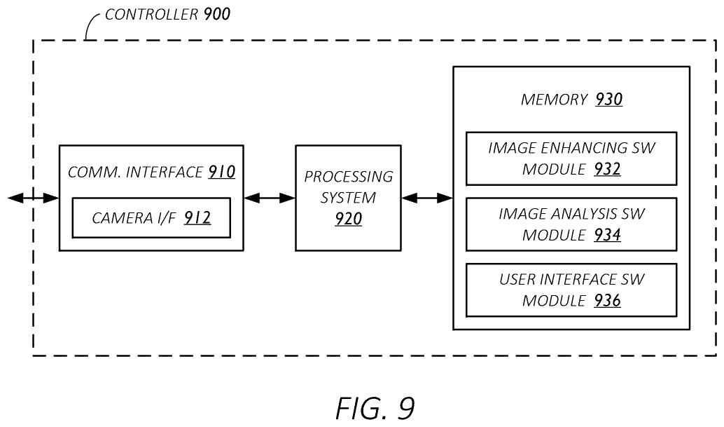

FIG. 9 shows a block diagram of an example controller 900 for a medical system, according to some implementations. In some implementations, the controller 900 may be one example of the image processing pipeline 500 of FIG. 5 or the control circuitry 302 of FIG. 3. More specifically, the controller 900 is configured to provide assistance with navigating a medical instrument within an anatomy based on images captured by a camera disposed on a distal end of the medical instrument.

The controller 900 includes a communication interface 910, a processing system 920, and a memory 930. The communication interface 910 is configured to communicate with one or more components of the medical system. More specifically, the communication interface 910 includes a camera interface (I/F) 912 for communicating with a camera associated with the medical system. In some implementations, the camera I/F 912 may receive image data captured by the camera disposed on the distal end of the instrument inserted within the anatomy.

The memory 930 may include a non-transitory computer-readable medium (including one or more nonvolatile memory elements, such as EPROM, EEPROM, Flash memory, or a hard drive, among other examples) that may store the following software (SW) modules: an image enhancing SW module 932 to infer enhanced image data from the received image data based on a neural network model trained to filter visual artifacts or obstructions from image data; an image analysis SW module 934 to extract information from the enhanced image data based on one or more image processing operations; and a user interface SW module 936 to generate a graphical user interface (GUI) for navigating the instrument within the anatomy based at least in part on the information extracted from the enhanced image data.

The processing system 920 may include any suitable one or more processors capable of executing scripts or instructions of one or more software programs stored in the controller 900 (such as in the memory 930). For example, the processing system 920 may execute the image enhancing SW module 932 to infer enhanced image data from the received image data based on a neural network model trained to filter visual artifacts or obstructions from image data. Further, the processing system 920 may execute the image analysis SW module 934 to extract information from the enhanced image data based on one or more image processing operations. Still further, the processing system 920 may execute the user interface SW module 936 to generate a GUI for navigating the instrument within the anatomy based at least in part on the information extracted from the enhanced image data.

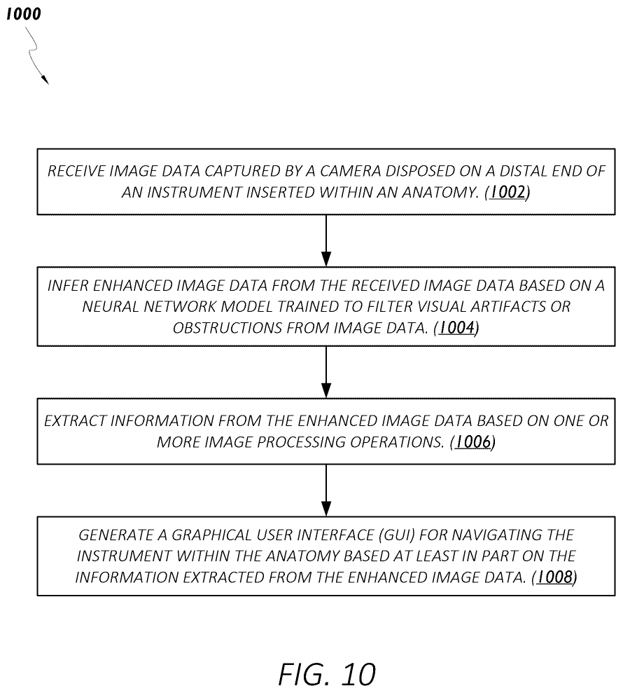

FIG. 10 shows an illustrative flowchart depicting an example operation 1000 for determining relative instrument positions, according to some implementations. In some implementations, the example operation 1000 may be performed by a controller for a medical system such as the controller 900 of FIG. 9.

The controller receives image data captured by a camera disposed on a distal end of an instrument inserted within an anatomy (1002). In some implementations, the anatomy may be a lung. In some other implementations, the anatomy may be a kidney. The controller infers enhanced image data from the received image data based on a neural network model trained to filter visual artifacts or obstructions from image data (1004). In some implementations, the neural network model may include a generative image inpainting model, an AI-based super resolution model, a generative style transfer model, or a NeRF or Gaussian splatting model. In some implementations, the visual artifacts may include blur, lighting variations, specular reflections, camera saturation, over-exposure, or under-exposure. In some implementations, the obstructions may include mucus, blood, stone dust or fragments, bubbles, or other medical instruments.

The controller extracts information from the enhanced image data based on one or more image processing operations (1006). In some implementations, the extracted information may include a position or orientation of the medical instrument. In some other implementations, the extracted information may include a shape, boundary, eccentricity, texture, or position of a feature of the anatomy. The controller further generates a graphical user interface (GUI) for navigating the instrument within the anatomy based at least in part on the information extracted from the enhanced image data (1008). In some implementations, the GUI may include an anatomical map indicating a spatial relationship between the instrument and a target within the anatomy. In some other implementations, the enhanced image data may be displayed as a live camera view in the GUI.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure.

Thus, the claims are not intended to be limited to the implementations shown herein, but are to be accorded the widest scope consistent with this disclosure, the principles and the novel features disclosed herein.

What is claimed is:

1. A method for controlling a medical system, comprising:
receiving image data captured by a camera disposed on a distal end of an instrument inserted within an anatomy;
inferring enhanced image data from the received image data based on a neural network model trained to filter visual artifacts or obstructions from image data;
extracting information from the enhanced image data based on one or more image processing operations; and
generating a graphical user interface (GUI) for navigating the instrument within the anatomy based at least in part on the information extracted from the enhanced image data.

2. The method of claim 1, wherein the visual artifacts include blur, lighting variations, specular reflections, camera saturation, over-exposure, or under-exposure.

3. The method of claim 1, wherein the obstructions include mucus, blood, stone dust or fragments, bubbles, or other medical instruments.

4. The method of claim 1, wherein the anatomy comprises a lung.

5. The method of claim 1, wherein the anatomy comprises a kidney.

6. The method of claim 1, wherein the neural network model comprises a generative image inpainting model, an artificial intelligence (AI)-based super resolution model, a generative style transfer model, or a Neural Radiance Field (NeRF) or Gaussian splatting model.

7. The method of claim 1, wherein the extracted information includes a position or orientation of the medical instrument.

8. The method of claim 1, wherein the extracted information includes a shape, boundary, eccentricity, texture, or position of a feature of the anatomy.

9. The method of claim 1, wherein the GUI includes an anatomical map indicating a spatial relationship between the instrument and a target within the anatomy.

10. The method of claim 1, wherein the enhanced image data is displayed as a live camera view in the GUI.

11. A controller for a medical system, comprising:
a processing system; and
a memory storing instructions that, when executed by the processing system, cause the controller to:
receive image data captured by a camera disposed on a distal end of an instrument inserted within an anatomy;
infer enhanced image data from the received image data based on a neural network model trained to filter visual artifacts or obstructions from image data;
extract information from the enhanced image data based on one or more image processing operations; and
generate a graphical user interface (GUI) for navigating the instrument within the anatomy based at least in part on the information extracted from the enhanced image data.

12. The controller of claim 11, wherein the visual artifacts include blur, lighting variations, specular reflections, camera saturation, over-exposure, or under-exposure.

13. The controller of claim 11, wherein the obstructions include mucus, blood, stone dust or fragments, bubbles, or other medical instruments.

14. The controller of claim 11, wherein the anatomy comprises a lung.

15. The controller of claim 11, wherein the anatomy comprises a kidney.

16. The controller of claim 11, wherein the neural network model comprises a generative image inpainting model, an artificial intelligence (AI)-based super resolution model, a generative style transfer model, or a Neural Radiance Field (NeRF) or Gaussian splatting model.

17. The controller of claim 11, wherein the extracted information includes a position or orientation of the medical instrument.

18. The controller of claim 11, wherein the extracted information includes a shape, boundary, eccentricity, texture, or position of a feature of the anatomy.

19. The controller of claim 11, wherein the GUI includes an anatomical map indicating a spatial relationship between the instrument and a target within the anatomy.

20. The controller of claim 11, wherein the enhanced image data is displayed as a live camera view in the GUI.
